(19) United States
(12) Patent Application Publication
(10) Pub. No.: US 2026/0076577 A1
(43) Pub. Date: Mar. 19, 2026

(54) DEVICE AND METHOD FOR DETERMINING OCCURRENCE OF CYBER SICKNESS

(71) Applicants: HYUNDAI MOTOR COMPANY, Seoul (KR); KIA CORPORATION, Seoul (KR)

(72) Inventors: Ki Chang KIM, Gyeonggi-do (KR); Dong Chul PARK, Gyeonggi-do (KR)

(73) Assignees: HYUNDAI MOTOR COMPANY, Seoul (KR); KIA CORPORATION, Seoul (KR)

(21) Appl. No.: 19/036,946

(22) Filed: Jan. 24, 2025

(30) Foreign Application Priority Data
Sep. 13, 2024 (KR) 10-2024-0125240

(51) Int. Cl. A61B 5/0205; A61B 3/113; A61B 5/00; A61B 5/024; A61B 5/374

(52) U.S. Cl. CPC A61B 5/0205; A61B 3/113; A61B 5/374; A61B 5/6803; A61B 5/02405

(57) ABSTRACT

A device for determining occurrence of cyber sickness, includes a sensor unit configured to collect bio information of a user who experiences virtual-environment driving content through a head-mounted display (HMD) and a vibration seat, a first processing unit configured to determine a first score by analyzing eye movements of the user using the bio information, a second processing unit configured to determine a second score by analyzing heart rate variability (HRV) of the user using the bio information, a third processing unit configured to determine a third score by analyzing a brainwave pattern of the user using the bio information, and a fourth processing unit configured to determine a cyber sickness index (CSI) of the user using the first score, the second score, and the third score.

### CROSS-REFERENCE TO RELATED APPLICATION

The present application claims priority to Korean Patent Application No. 10-2024-0125240, filed on Sep. 13, 2024, the entire contents of which is incorporated herein for all purposes by this reference.

### BACKGROUND OF THE PRESENT DISCLOSURE

### Field of the Present Disclosure

The present disclosure relates to a device and method for determining occurrence of cyber sickness.

### Description of Related Art

Motion sickness or cyber sickness is kinesia that occurs during the use of virtual reality (VR), augmented reality (AR), video games, three-dimensional (3D) movies, and the like. This is caused by a mismatch between movement-related visual information and actual physical sensations, and symptoms may be similar to traditional motion sickness.

Lately, there has been an increase in consumption patterns in the emotional information and communication technology (ICT) field for detecting and perceiving users'emotions and providing services using information technology (IT) devices. Also, with the technological development and an increase in the supply of autonomous vehicles, the market of content which may be experienced in vehicles is rapidly expanding.

For the present reason, various studies are being conducted on cyber sickness which is caused by a mismatch between the visual and auditory senses during the experience of virtual content.

The information included in this Background of the present disclosure is only for enhancement of understanding of the general background of the present disclosure and may not be taken as an acknowledgement or any form of suggestion that this information forms the prior art already known to a person skilled in the art.

### BRIEF SUMMARY

Various aspects of the present disclosure are directed to providing a device and method for determining occurrence of cyber sickness which allow prediction and judgment about motion sickness caused by sensory conflict in a virtual environment.

According to various aspects of the present disclosure, there is provided a device for determining occurrence of cyber sickness, the device including a sensor unit configured to collect bio information of a user who experiences virtual-environment driving content through a head-mounted display (HMD) and a vibration seat, a first processing unit configured to determine a first score by analyzing eye movements of the user using the bio information, a second processing unit configured to determine a second score by analyzing heart rate variability (HRV) of the user using the bio information, a third processing unit configured to determine a third score by analyzing a brainwave pattern of the user using the bio information, and a fourth processing unit configured to determine a cyber sickness index (CSI) of the user using the first score, the second score, and the third score.

The first processing unit may be configured to determine the first score by analyzing whether vergence of the user's eyes matches accommodation of the eyes.

The second processing unit may be configured to determine the second score by analyzing a heart rate, a heart rate pattern, and a standard deviation of NN intervals (SDNN) value of the user.

The third processing unit may be configured to determine the third score by analyzing frequency changes of alpha waves, theta waves, and delta waves of the user.

The fourth processing unit may be configured to determine the CSI by adding the first score, the second score, and the third score.

The fourth processing unit may be configured to determine score-specific contribution factors by performing regression analysis on the first score, the second score, and the third score.

The fourth processing unit may be configured to determine the CSI by applying the contribution factors as weights of the first score, the second score, and the third score.

The virtual-environment driving content may include a virtual image and a virtual sound generated based on a driving environment of a vehicle driver.

The virtual sound may include a high-order ambisonics signal.

The sensor unit may include a first sensor provided in the HMD to detect the eye movements of the user, a second sensor provided in the vibration seat to measure a heart rate of the user, and a third sensor provided in the HMD to detect brainwaves of the user.

According to various aspects of the present disclosure, there is provided a method of determining occurrence of cyber sickness, the method including collecting, by a sensor unit, bio information of a user who experiences virtual-environment driving content through an HMD and a vibration seat, determining, by a first processing unit, a first score by analyzing eye movements of the user using the bio information, determining, by a second processing unit, a second score by analyzing HRV of the user using the bio information, determining, by a third processing unit, a third score by analyzing a brainwave pattern of the user using the bio information, and determining, by a fourth processing unit, a CSI of the user using the first score, the second score, and the third score.

The determining of the first score may include determining the first score by analyzing whether vergence of the user's eyes matches accommodation of the eyes.

The determining of the second score may include determining the second score by analyzing a heart rate, a heart rate pattern, and an SDNN value of the user.

The determining of the third score may include determining the third score by analyzing frequency changes of alpha waves, theta waves, and delta waves of the user.

The determining of the CSI may include determining the CSI by adding the first score, the second score, and the third score.

The determining of the CSI may include determining score-specific contribution factors by performing regression analysis on the first score, the second score, and the third score.

The determining of the CSI may include determining the CSI by applying the contribution factors as weights of the first score, the second score, and the third score.

The virtual-environment driving content may include a virtual image and a virtual sound generated based on a driving environment of a vehicle driver.

The virtual sound may include a high-order ambisonics signal.

The collecting of the bio information may include detecting, by a first sensor provided in the HMD, the eye movements of the user, measuring, by a second sensor provided in the vibration seat, a heart rate of the user, and detecting, by a third sensor provided in the HMD, brainwaves of the user.

A device and method for determining occurrence of cyber sickness according to various exemplary embodiments allow prediction and judgment about motion sickness caused by sensory conflict in a virtual environment.

Furthermore, it is possible to predict whether a user has motion sickness and the degree of motion sickness when experiencing virtual content based on a vehicle driving environment.

The methods and apparatuses of the present disclosure have other features and advantages which will be apparent from or are set forth in more detail in the accompanying drawings, which are incorporated herein, and the following Detailed Description, which together serve to explain certain principles of the present disclosure.

### BRIEF DESCRIPTION OF THE DRAWINGS

FIG. 1 is a diagram illustrating an operating environment of a device for determining occurrence of cyber sickness according to an exemplary embodiment of the present disclosure.

FIG. 2 and FIG. 3 are diagrams illustrating the concept of virtual-environment driving content according to an exemplary embodiment of the present disclosure.

FIG. 4 is a block diagram of a device for determining occurrence of cyber sickness according to an exemplary embodiment of the present disclosure.

FIG. 5 is a diagram illustrating operations of the device for determining occurrence of cyber sickness according to an exemplary embodiment of the present disclosure.

FIG. 6 and FIG. 7 are diagrams illustrating a sensor unit according to an exemplary embodiment of the present disclosure.

FIG. 8 is a diagram illustrating an operation of a first processing unit according to an exemplary embodiment of the present disclosure.

FIG. 9 is a diagram illustrating an operation of a second processing unit according to an exemplary embodiment of the present disclosure.

FIG. 10 is a diagram illustrating an operation of a third processing unit according to an exemplary embodiment of the present disclosure.

FIG. 11, FIG. 12, FIG. 13 and FIG. 14 are diagrams illustrating an operation of a fourth processing unit according to an exemplary embodiment of the present disclosure.

FIG. 15 is a flowchart of a method of determining occurrence of cyber sickness according to an exemplary embodiment of the present disclosure.

It may be understood that the appended drawings are not necessarily to scale, presenting a somewhat simplified representation of various features illustrative of the basic principles of the present disclosure. The specific design features of the present disclosure as included herein, including, for example, specific dimensions, orientations, locations, and shapes will be determined in part by the particularly intended application and use environment.

In the figures, reference numbers refer to the same or equivalent portions of the present disclosure throughout the several figures of the drawing.

### DETAILED DESCRIPTION

Reference will now be made in detail to various embodiments of the present disclosure(s), examples of which are illustrated in the accompanying drawings and described below. While the present disclosure(s) will be described in conjunction with exemplary embodiments of the present disclosure, it will be understood that the present description is not intended to limit the present disclosure(s) to those exemplary embodiments of the present disclosure. On the other hand, the present disclosure(s) is/are intended to cover not only the exemplary embodiments of the present disclosure, but also various alternatives, modifications, equivalents and other embodiments, which may be included within the spirit and scope of the present disclosure as defined by the appended claims.

Hereinafter, various exemplary embodiments of the present disclosure will be described in detail with reference to the accompanying drawings.

However, the technical spirit of the present disclosure is not limited to the disclosed exemplary embodiments but may be implemented in a variety of different forms. Within the technical spirit of the present disclosure, one or more components of embodiments may be selectively combined or substituted and used.

Also, terms (including technical and scientific terms) may be interpreted with meanings that may be generally understood by those of ordinary skill in the art unless clearly and particularly defined and described. Generally used terms, such as terms defined in a dictionary, may be interpreted in consideration of the meaning in the context of the related technology.

Furthermore, terms used in embodiments of the present disclosure are for describing the exemplary embodiments and are not intended to limit the present disclosure.

In the present specification, a singular form may include the plural form unless specifically stated in the phrase, and when described as “at least one (or one or more) of A, B, and C,” it may include one or more of all combinations thereof.

Terms such as “first,” “second,” “A,” “B,” “(a),” “(b),” and the like may be used in describing components of embodiments of the present disclosure.

These terms are only for distinguishing a component from others, and the nature, turn, sequence, or the like of the component is not limited by the terms.

When a component is referred to as being “connected,” “coupled,” or “interconnected,” to another component, the component may not only be directly connected, coupled, or interconnected to the other component, but may also be “connected,” “coupled,” or “interconnected” to the other component via yet another component therebetween.

Furthermore, when a component is referred to as being formed or disposed “on (above)” or “under (below)” another component, the two components may be in direct contact with each other or one or more other components may be formed or disposed therebetween. Furthermore, when a component is expressed as being “on (above)” or “under (below)” another component, the component may not be only in an upward direction but also in a downward direction based on the other component.

Hereinafter, various exemplary embodiments will be described in detail with reference to the accompanying drawings. Throughout the drawings, like components will be assigned like reference numerals, and the detailed description thereof will be omitted.

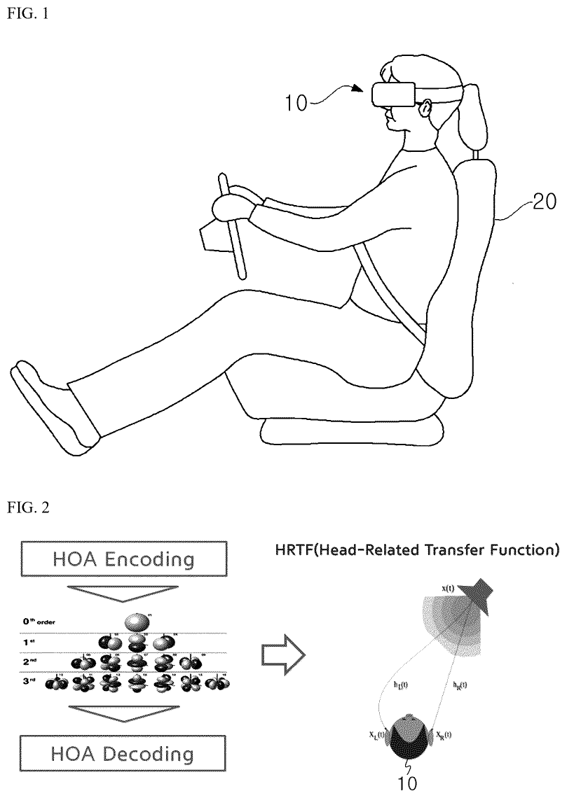

FIG. 1 is a diagram illustrating an operating environment of a device for determining occurrence of cyber sickness according to an exemplary embodiment of the present disclosure. Referring to FIG. 1, a device 100 for determining occurrence of cyber sickness according to various exemplary embodiments of the present disclosure may be applied to an autonomous vehicle user who experiences virtual-environment content while wearing a head-mounted display (HMD) in a metaverse environment or the like, to quantitatively determine whether motion sickness occurs and the degree of motion sickness.

According to an exemplary embodiment of the present disclosure, the HMD 10 may be a display device which is disposed in the autonomous vehicle and worn on the user's head. The HMD 10 may be mainly used in virtual reality (VR) and augmented reality (AR) applications and disposed in front of the user's eyes to provide an immersive visual experience.

The HMD 10 may include a display panel that provides an image to the user using two small screens or one large screen, and a lens which is placed between the display and the user's eyes to adjust the image in accordance with the eyes.

The HMD 10 may track the user's head movement using head tracking technology implemented through a gyroscope, an accelerometer, a magnetic field sensor, and the like and adjust the view of a screen.

The HMD 10 may provide virtual-environment driving content to the user wearing the HMD 10. According to an exemplary embodiment of the present disclosure, the virtual-environment driving content may include a virtual image and a virtual sound generated based on the driving environment of a vehicle driver.

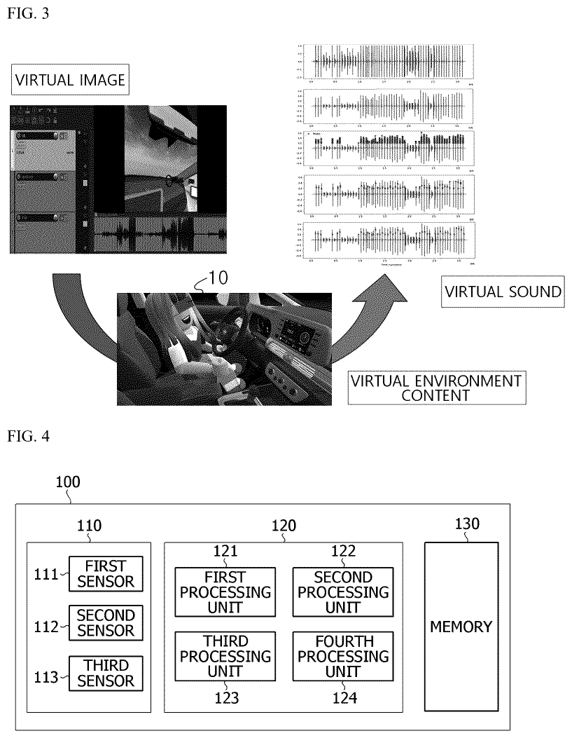

FIG. 2 and FIG. 3 are diagrams illustrating the concept of virtual-environment driving content according to an exemplary embodiment of the present disclosure. Referring to FIG. 2 and FIG. 3, a virtual image may be data for visualizing an external background that a vehicle driver or passenger may actually experience through a vehicle window while a vehicle travels.

Also, virtual sound may include various noises and music that the vehicle driver or passenger may recognize in the vehicle while the vehicle travels.

For example, the virtual sound may include a high-order ambisonics signal.

According to an exemplary embodiment of the present disclosure, virtual-environment driving content can reduce scattering of the fidelity of virtual images and virtual sounds by applying sound perception differences resulting from the structure and shape of the head and ears to head-related transfer function (HRTF) logic having the concept of a stereo sound implementation filter based on user personalization. High-order ambisonics is a method of reproducing stereoscopic sound by arranging speaker devices in a shape of a sphere centered on a listener. According to high-order ambisonics, it is possible to improve the heterogeneity caused by sound inaccuracy for images and implement lifelike sounds.

The HRTF logic may transform sound waves which travel from a sound source located at a specific azimuth and elevation angle toward a listener, into sound waves which have characteristics necessary for directional perception due to the head shape, auricle structure, shoulder shape, and the like of the individual that the sound waves pass through to reach the listener's ears. The HRTF logic may measure such characteristics that cause these changes and express the characteristics in a form of a transfer function. Since a body shape significantly varies for each individual, each person includes a different HRTF. Accordingly, an HRTF customized for each user is necessary to accurately use HRTF logic. However, to obtain HRTF data, it is necessary to take measurements at a determined azimuth and altitude. Since equipment for taking measurements is complex and measuring takes a long time, it is impossible to measure HRTFs of all users in practice. Therefore, in general, signal processing for binaural sound source manufacturing may be performed using HRTF characteristics of a standard Knowles Electronics Manikin for Acoustic Research (KEMAR) dummy head or a public HRTF database (DB) of test subjects provided by a research institute such as Acoustics Research Institute (ARI), Center for Image Processing and Integrated Computing (CIPIC), Institut de Recherche et Coordination Acoustique/Musique (IRCAM), or the like.

High-order ambisonics is a technology for applying a panning technique for adjusting a sound position in a virtual space not only to a spherical surface but also to the inside or outside of the spherical surface. Spherical waves may be expressed as the sum of spherical harmonics. Using this, it is possible to play sound waves represented by spherical harmonics through each speaker and add the sound waves to generate the same sound wave as is output by a virtual sound source desired by a user. Low-order ambisonics which utilize a small number of spherical harmonics do not generate a large sound field but generate a very small sweet spot, a position where a user can feel an exact virtual sound field. To overcome this, high-order ambisonics technology is applied. The minimum number of speakers required for implementing n-order ambisonics technology may be defined as (n+1)2.

When the virtual-environment driving content is executed, the user perceives a virtual image and a virtual sound, and the device for determining occurrence of cyber sickness according to various exemplary embodiments of the present disclosure may perform bio information analysis to analyze a correlation between sensory conflict and motion sickness. The device 100 for determining occurrence of cyber sickness can determine a quantitative motion sickness judgment index by analyzing virtual environment fidelity of the user who experiences the virtual-environment driving content while sitting on a vibration seat 20 and wearing the HMD 10.

FIG. 4 is a block diagram of a device for determining occurrence of cyber sickness according to an exemplary embodiment of the present disclosure, and FIG. 5 is a diagram illustrating operations of the device for determining occurrence of cyber sickness according to an exemplary embodiment of the present disclosure. Referring to FIG. 4 and FIG. 5, the device 100 for determining occurrence of cyber sickness according to various exemplary embodiments of the present disclosure may include a sensor unit 110, a processor 120, and a memory 130. The device 100 for determining occurrence of cyber sickness may be implemented in a vehicle to communicate with electronic portions in the vehicle.

The sensor unit 110 may be provided in an HMD and a vibration seat to collect bio information of a user who experiences virtual-environment driving content.

The sensor unit 110 may include a first sensor 111 provided in the HMD to detect eye movements of the user, a second sensor 112 provided in the vibration seat to measure a heart rate of the user, and a third sensor 113 provided in the HMD to detect brainwaves of the user.

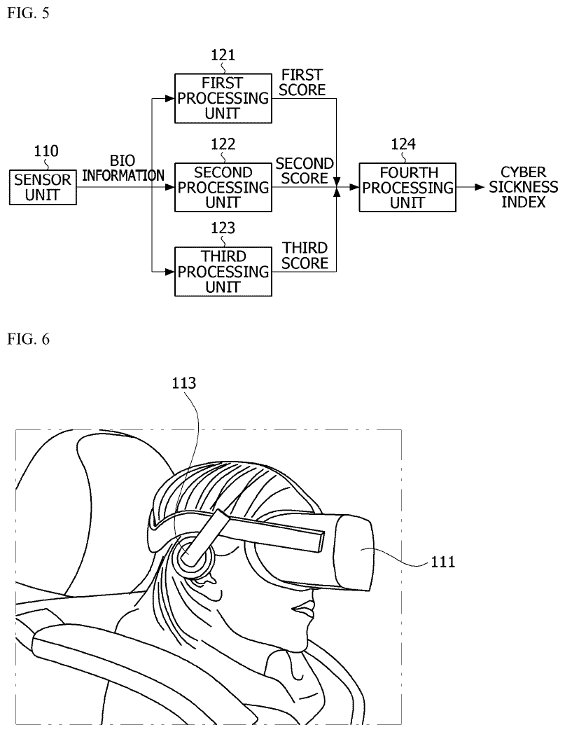

FIG. 6 and FIG. 7 are diagrams illustrating a sensor unit according to an exemplary embodiment of the present disclosure. Referring to FIG. 6 and FIG. 7 together, the first sensor 111 may be provided in a goggle portion of the HMD 10. A high-speed camera or infrared (IR) camera, as an exemplary embodiment of the first sensor 111 may be used to image a user's eyes in real time. The camera may be integrated within the HMD or attached to the external surface of the HMD to accurately track the positions and movements of the eyes. A light source of the first sensor 111 may illuminate the eyes using an IR light-emitting diode (LED) or other nonvisible rays to more clarify a reflection pattern of the eyes, which allows the camera to track movements more accurately. Image processing software of the first sensor 111 may be configured to determine the positions and sizes of the pupils by analyzing a captured image to track movements of the eyes and determine what the user is looking at.

The second sensor 112 may be provided in the vibration seat 20. The second sensor 112 may include at least one of a photoplethysmography (PPG) sensor, an electrocardiography (ECG) sensor, and an acoustic sensor.

The PPG sensor may include a light-emitting diode (LED) and an optical sensor. The PPG sensor may measure the amount of blood using light. In the PPG sensor, the LED may radiate light to the skin, and a heart rate may be measured based on the principle that the amount of light reflected through the skin and blood vessels varies depending on the amount of blood.

The ECG sensor may include electrodes attached to the skin and a signal processing circuit. The ECG sensor may detect an electrical action which occurs when the heart beats, through the electrodes attached to the skin.

The acoustic sensor may include a highly sensitive microphone and a signal processing system. The acoustic sensor may record heartbeat sounds using the microphone and analyze the heartbeat sounds to measure a heart rate.

The third sensor 113 may be provided in an earring portion of the HMD 10. The third sensor 113 may include at least one of an electroencephalography (EEG) sensor, a magnetoencephalography (MEG) sensor, and a functional near-infrared spectroscopy (fNIRS) sensor.

The EEG sensor may include electrodes, an amplifier, and a signal processing system. The EEG sensor may measure an electrical action of the brain through the electrodes attached to the scalp, detect an electrical change caused by the activity of neurons, and convert the electrical change into a signal.

The MEG sensor may include a magnetic sensor and a signal processing system. The MEG sensor may detect a fine magnetic change using a highly sensitive magnetic sensor, such as a superconducting quantum interference device (SQUID) or the like, and convert the magnetic change into a brainwave signal.

The fNIRS sensor may include a near-infrared light source, a light sensor, and a signal processing system. The fNIRS sensor may measure blood oxygen saturation in the brain using near-infrared rays and detect brainwaves using the principle that oxygenated blood and deoxygenated blood absorb near-infrared light to different degrees.

According to an exemplary embodiment of the present disclosure, each component may include a function and capability other than those described above, and additional components other than those described below may be included. Also, according to an exemplary embodiment of the present disclosure, each component may be implemented using one or more devices that are physically separated, or a combination of one or more processors 120 or the one or more processors 120 and software. Unlike the example shown in the drawings, each component may not be clearly distinguished in terms of detailed operations.

The device 100 according to various exemplary embodiments of the present disclosure may be implemented in a logic circuit as hardware, firmware, or software, or a combination thereof or implemented using a general-use computer or a special-purpose computer. The device may be implemented using a hardwired device, a field programmable gate array (FPGA), an application specific integrated circuit (ASIC), or the like. Also, the device may be implemented as a system on chip (SoC) including the one or more processors 120 and a controller.

Furthermore, the device 100 may be provided in a computing device or a server in which hardware elements are provided, in a form of software, hardware, or a combination thereof. The computing device or server may be various devices including all or some of a communication device, such as a communication modem or the like, for communicating with various devices or a wired or wireless communication network, a memory 130 for storing data for executing a program, a microprocessor for executing the program to perform a computation and give a command, and the like.

The memory 130 may include a DB. The memory 130 may be a non-transitory storage medium that stores instructions executed by the processor 120. The memory 130 may include at least one of storage media such as a random access memory (RAM), a static random access memory (SRAM), a read-only memory (ROM), a programmable read-only memory (PROM), an electrically erasable and programmable ROM (EEPROM), an erasable and programmable ROM (EPROM), a Hard Disk Drive (HDD), a solid state disk (SSD), an embedded multimedia card (eMMC), a universal flash storage (UFS), a web storage, and the like.

According to an exemplary embodiment of the present disclosure, a first processing unit 121, a second processing unit 122, a third processing unit 123, and a fourth processing unit 124 may be implemented through the same process. For convenience of description, operations of each of the components will be separately described below.

Herein, in an exemplary embodiment of the present disclosure, the first processing unit 121, the second processing unit 122, the third processing unit 123, and the fourth processing unit 124 may be implemented as separate processors. Alternatively, t the first processing unit 121, the second processing unit 122, the third processing unit 123, and the fourth processing unit 124 may be implemented as a single processor.

The processor 120 may include at least one of processing devices such as an ASIC, a digital signal processor (DSP), a programmable logic device (PLD), an FPGA, a central processing unit (CPU), a microcontroller, a microprocessor, and the like.

The first processing unit 121 may be configured to determine a first score by analyzing the user's eye movements using bio information.

The first processing unit 121 may be configured to determine the first score by analyzing whether vergence of the user's eyes matches accommodation of the eyes.

Vergence may be a movement of both eyes rotating in different directions to look at the same object. This is one important visual function that allows both eyes to perceive depth in cooperation with each other.

Vergence allows depth perception and stereopsis. Since the eyes look at an object from slightly different angles, the brain can integrate the present information to perceive three-dimensional (3D) depth.

Accommodation may be a process in which the lens of an eye thickens or thins to focus on an object.

Vergence and accommodation are closely linked and may work together to optimize a regulatory function of the visual system. For example, when a person looks at a nearby object, vergence causes both eyes to converge to the center portion to focus on the object, while accommodation causes the lenses to thicken so that light is accurately focused on the retinas.

Therefore, the two processes work together to allow a person to look at an object clearly and accurately. In the present way, it is possible to clearly perceive objects at various distances and effectively process visual information in daily life.

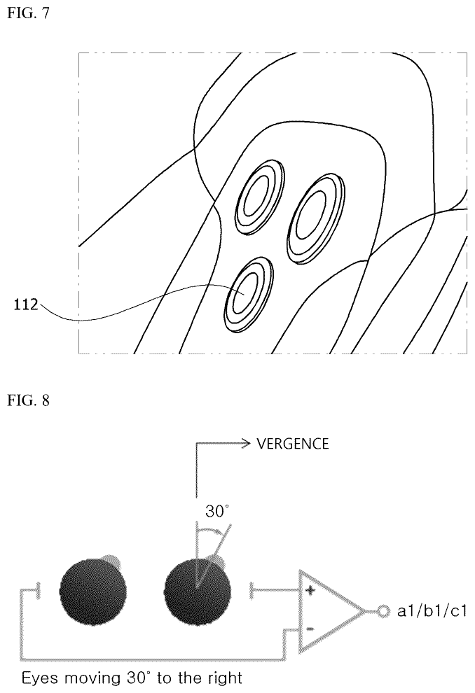

FIG. 8 is a diagram illustrating an operation of the first processing unit 121 according to an exemplary embodiment of the present disclosure. Additionally referring to FIG. 8, it is possible to see a process of determining occurrence of motion sickness caused by brain stimulation based on a mismatch between vergence and accommodation to determine occurrence of cyber sickness through eye movement analysis. In other words, when vergence in which both eyes move in opposite directions to look at a faraway object does not match accommodation of focusing on a display, cyber sickness occurs.

For example, when vergence matches accommodation of the eyes, the first processing unit 121 may be configured to determine that it is a normal state with a low risk level of cyber sickness, and determine a first score to be a1. When vergence does not match accommodation of the eyes, the first processing unit 121 may be configured to determine that it is a dangerous stage with a high risk level of cyber sickness, and determine the first score to be c1. When matching of vergence and accommodation of the eyes is in a border region, the first processing unit 121 may be configured to determine that it is a border stage with a medium risk level of cyber sickness, and determine the first score to be b1. According to an exemplary embodiment of the present disclosure, a1, b1, and c1 all are positive integers and satisfy a1>b1>c1.

The second processing unit 122 may be configured to determine a second score by analyzing the user's heart rate variability (HRV) using the bio information. The second processing unit 122 may be configured to determine the second score by analyzing the user's heart rate, heart rate pattern, and a standard deviation of NN intervals (SDNN) value.

The heart rate may be the number of beats of the heart in one minute and may be measured in units of beats per minute (bpm).

The heart rate pattern represents changes of the heartbeat over time and may include a heart rhythm, periodicity, and variability. A heart in normal rhythm (normal sinus rhythm) may beat at regular intervals, and a heart in irregular rhythm (arrhythmia) may beat at irregular intervals.

SDNN is one of indicators for measuring HRV. Here, “NN intervals” are normal R-R intervals, and an R-R interval may be the time between R waves (R peaks) of two consecutive heartbeats in ECG. This reflects the heart's autonomic nervous system activity. For example, a high SDNN indicates that the autonomic nervous system is active and heart health is good, while a low SDNN indicates that the autonomic nervous system is underactive or may include a heart health problem.

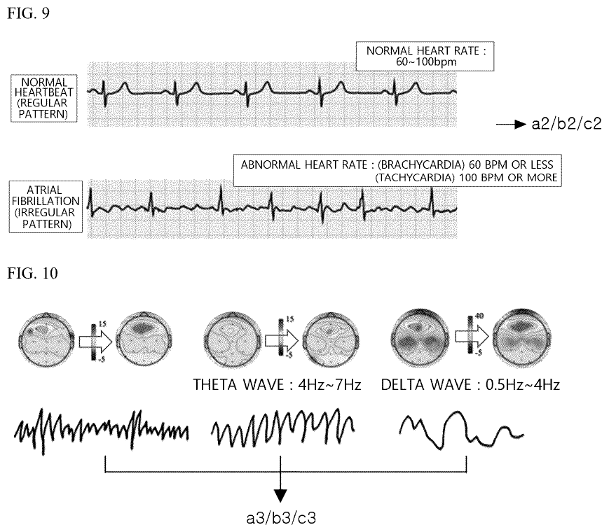

FIG. 9 is a diagram illustrating an operation of the second processing unit 122 according to an exemplary embodiment of the present disclosure. Additionally referring to FIG. 9, to determine occurrence of motion sickness through HRV analysis, it is possible to identify a factor that causes motion sickness when the heart rate is 60 to 100 and the heart rate pattern is irregular.

For example, when the heart rate is 60 bpm to 100 bpm, the heart rate pattern is regular, and the SDNN value is 3 or larger, the second processing unit 122 may be configured to determine that it is a normal state with a low risk level of cyber sickness, and determine a second score to be a2. When the heart rate deviates from the range from 60 bpm to 100 bpm, the heart rate pattern is irregular, or the SDNN value is smaller than 3, the second processing unit 122 may be configured to determine that it is a border stage with a medium risk level, and determine the second score to be b2. When two or more of the following are true: the heart rate deviates from the range from 60 bpm to 100 bpm, the heart rate pattern is irregular, and the SDNN value is smaller than 3, the second processing unit 122 may be configured to determine that it is a dangerous stage with a high risk level of cyber sickness, and determine the second score to be c2. According to an exemplary embodiment of the present disclosure, a2, b2, and c2 all are positive integers and satisfy a2>b2>c2.

The third processing unit 123 may be configured to determine a third score by analyzing the user's brainwave pattern using the bio information. The third processing unit 123 may be configured to determine the third score by analyzing frequency changes of alpha waves, theta waves, and delta waves of the user.

Brainwaves may be categorized as delta waves, theta waves, alpha waves, beta waves, and gamma waves by frequency. Brainwave patterns that are closely associated with motion sickness are beta waves, theta waves, and delta waves, and their characteristics are as follows.

Beta waves have a frequency band of 15 Hz to 18 Hz, and correspond to an increased level of arousal when a person needs to focus his or her attention. Beta waves which are associated with concentration and tension may be used to analyze changes in human perception and emotion that are caused by stimuli from external factors. Beta waves are activated when a person perceives and judges an object and when an external information stimulus is complex in a perception and cognition process.

Theta waves have a frequency band of 4 Hz to 7 Hz and may represent a drowsiness indicator associated with motion sickness.

Delta waves have a frequency band of 0.5 Hz to 4 Hz and may represent a dizziness indicator associated with motion sickness.

FIG. 10 is a diagram illustrating an operation of the third processing unit 123 according to an exemplary embodiment of the present disclosure. Additionally referring to FIG. 10, it is possible to see that an algorithm based on analysis of brainwave frequency changes and brainwave pattern images may be utilized to determine occurrence of cyber sickness.

For example, when frequency changes of alpha waves associated with relaxation, theta waves associated with drowsiness, and delta waves associated with unconsciousness are within a preset range, that is, constant, the third processing unit 123 may be configured to determine that it is a normal state with a low risk level of cyber sickness, and determine a third score to be a3. When two or less of alpha waves, theta waves, and delta waves have frequency changes deviating from the preset range, the third processing unit 123 may be configured to determine that it is a dangerous stage with a high risk level of cyber sickness, and determine a third score to be b3. When frequency changes of alpha waves, theta waves, and delta waves deviate from the preset range, the third processing unit 123 may be configured to determine that it is a dangerous stage with a high risk level of cyber sickness, and determine a third score to be c3. According to an exemplary embodiment of the present disclosure, a3, b3, and c3 all are positive integers and satisfy a3>b3>c3.

The fourth processing unit 124 may be configured to determine a cyber sickness index (CSI) of the user using the first score, the second score, and the third score. The fourth processing unit 124 may be configured to determine the CSI by adding the first score, the second score, and the third score.

The fourth processing unit 124 may be configured to determine score-specific contribution factors through regression analysis of the first score, the second score, and the third score.

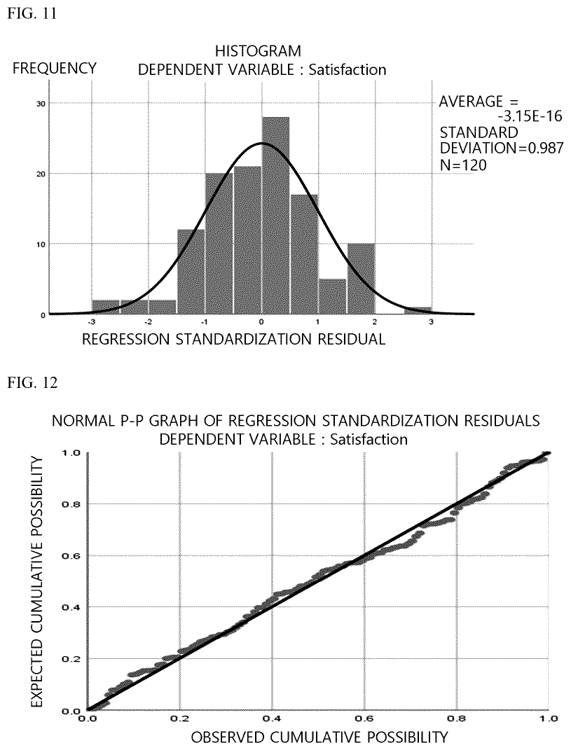

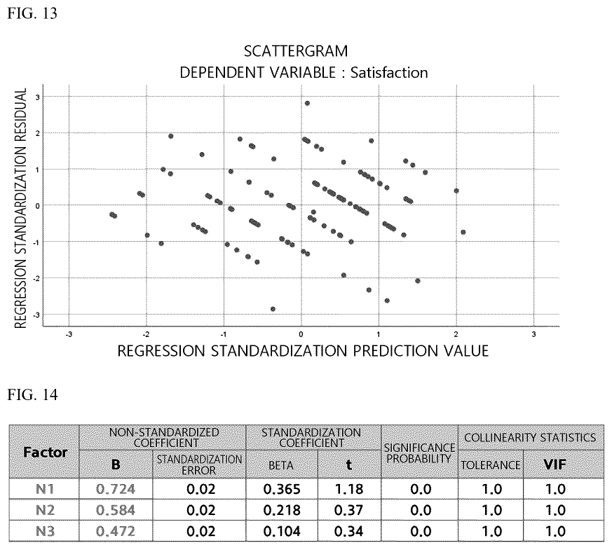

FIG. 11, FIG. 12, FIG. 13 and FIG. 14 are diagrams illustrating an operation of the fourth processing unit 124 according to an exemplary embodiment of the present disclosure. Additionally referring to FIG. 11, FIG. 12, FIG. 13 and FIG. 14, the fourth processing unit 124 may be configured to determine contribution factors N1, N2, and N3 through regression analysis and statistical analysis of a user's bio information evaluation. In statistics, regression analysis is an analytical method in which a model between two observed continuous variables is induced and then conformity is measured.

According to an exemplary embodiment of the present disclosure, causes of eye movements A, HRV B, and a brainwave pattern C and a consequence of cyber sickness caused by sensory conflict are continuous concepts, and regression may be described as the return of the relationship between variables to the mean of a generalized linear relationship.

The fourth processing unit 124 may be configured to determine a correlation equation through variable selection and model diagnostics. Variables may be categorized into independent variables and dependent variables. An independent variable is a variable which is used to predict or describe a result and includes an effect on another variable, while a dependent variable is a variable of which a result varies depending on a value of an independent variable, that is, a variable to be predicted through regression analysis. A regression model is an equation that models the relationship between an independent variable and a dependent variable and may be determined through regression analysis.

The histogram of FIG. 11 is a graphical representation of a frequency table in which the horizontal axis indicates motion sickness variables and the perpendicular axis indicates frequency. The histogram may be used to analyze dependent variable satisfaction.

In FIG. 12, the fourth processing unit 124 can minimize computational errors that occur when there are many independent variables by analyzing correlation between an observed cumulative probability and an expected cumulative probability through check of a regression standardization residual normal P-P graph.

The fourth processing unit 124 of FIG. 13 may display regression standardization prediction values and regression standardization residuals as points in a Cartesian coordinate system using a scattergram and analyze the relationship between the two variables.

In other words, the fourth processing unit 124 may analyze a pattern of each score using the histogram, determine a slope of a normal P-P graph, and derive a correlation equation through scattering using a scattergram.

Referring to FIG. 14, the fourth processing unit 124 may be configured to determine contribution factors through statistical analysis. According to an exemplary embodiment of the present disclosure, the contribution factors may not be fixed values and may change in accordance with analysis and a setting.

The fourth processing unit 124 may be configured to determine a standardization coefficient by multiplying standard deviation ratios of X and Y in a non-standardized coefficient of regression analysis. The fourth processing unit 124 may remove the fixed variable “a” from a basic equation which is expressed as “Y=a+B1X1+B2X2,” determine a value of a non-standardized coefficient B among the three other variables, and determine contribution factors as values of N1, N2, and N3.

The fourth processing unit 124 may be configured to determine a CSI by adding the first score, the second score, and the third score to which the contribution factors are applied as weights. For example, the fourth processing unit 124 may be configured to determine the CSI in accordance with Equation 1 below.

$$CSI = N 1 A + N 2 B + N 3 C \qquad [ Equation 1 ]$$

In Equation 1, A is a first score, B is a second score, C is a third score, N1 is a contribution factor of the first score, N2 is a contribution factor of the second score, and N3 is a contribution factor of the third score.

According to Equation 1, it is possible to determine the degree of motion sickness by correlating eye movements, HRV, and a brainwave pattern. The fourth processing unit 124 may be configured to determine the CSI to be a severe motion sickness stage, a border stage, or a favorable stage.

For example, even when eye movements correspond to a score of 1 (cyber sickness), a brainwave pattern corresponds to a score of 1 (cyber sickness), and HRV corresponds to a score of 3 (normal), a CSI may indicate severe motion sickness in accordance with the contribution factors.

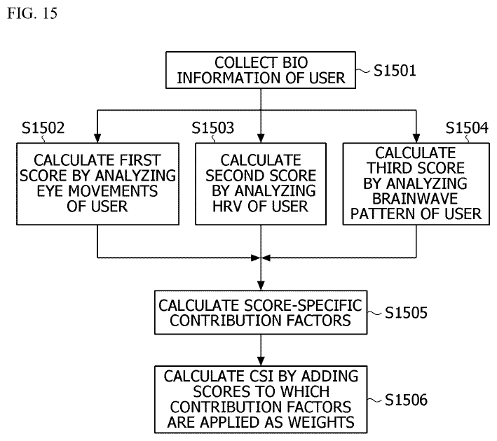

FIG. 15 is a flowchart of a method of determining occurrence of cyber sickness according to an exemplary embodiment of the present disclosure.

Referring to FIG. 15, a sensor unit collects bio information of a user who experiences virtual-environment driving content through an HMD and a vibration seat. The collected bio information may include the user's eye movements, heart rate, and brainwaves. The three kinds of bio information may be measured in the same time period within a certain error range (S1501).

Subsequently, a processor is configured to determine a first score by analyzing the user's eye movements using the bio information (S1502).

A second processing unit is configured to determine a second score by analyzing the user's HRV using the bio information (S1503).

A third processing unit is configured to determine a third score by analyzing the user's brainwaves using the bio information (S1504).

Operations of determining the first, second, and third scores may be simultaneously performed, or any one score may be determined before other scores.

Subsequently, a fourth processing unit may be configured to determine score-specific contribution factors through regression analysis of the first score, the second score, and the third score (S1505).

Subsequently, the fourth processing unit is configured to determine a CSI by adding the first score, the second score, and the third score to which the contribution factors are applied as weights (S1506).

As used in an exemplary embodiment of the present disclosure, the term “unit” refers to software or a hardware component, such as an FPGA or an ASIC, and a unit is configured to perform certain roles. However, a unit is not limited to software or hardware. A unit may be configured to be in an addressable storage medium or operate one or more processors. Accordingly, as an exemplary embodiment of the present disclosure, a unit includes components, such as software components, object-oriented software components, class components, and task components, processes, functions, attributes, procedures, subroutines, segments of program code, drivers, firmware, microcode, circuits, data, DBs, data structures, tables, arrays, and variables. Functionality provided within components and units may be combined into a smaller number of components and units or subdivided into additional components and units. Furthermore, components and units may be implemented to operate one or more CPUs in a device or secure multimedia card.

In various exemplary embodiments of the present disclosure, each operation described above may be performed by a control device, and the control device may be configured by a plurality of control devices, or an integrated single control device.

In various exemplary embodiments of the present disclosure, the memory and the processor may be provided as one chip, or provided as separate chips.

In various exemplary embodiments of the present disclosure, the scope of the present disclosure includes software or machine-executable commands (e.g., an operating system, an application, firmware, a program, etc.) for enabling operations according to the methods of various embodiments to be executed on an apparatus or a computer, a non-transitory computer-readable medium including such software or commands stored thereon and executable on the apparatus or the computer.

In various exemplary embodiments of the present disclosure, the control device may be implemented in a form of hardware or software, or may be implemented in a combination of hardware and software.

Software implementations may include software components (or elements), object-oriented software components, class components, task components, processes, functions, attributes, procedures, subroutines, program code segments, drivers, firmware, microcode, data, database, data structures, tables, arrays, and variables. The software, data, and the like may be stored in memory and executed by a processor. The memory or processor may employ a variety of means well known to a person having ordinary knowledge in the art.

Furthermore, the terms such as “unit”, “module”, etc. included in the specification mean units for processing at least one function or operation, which may be implemented by hardware, software, or a combination thereof.

In the flowchart described with reference to the drawings, the flowchart may be performed by the controller or the processor. The order of operations in the flowchart may be changed, a plurality of operations may be merged, or any operation may be divided, and a specific operation may not be performed. Furthermore, the operations in the flowchart may be performed sequentially, but not necessarily performed sequentially. For example, the order of the operations may be changed, and at least two operations may be performed in parallel.

Hereinafter, the fact that pieces of hardware are coupled operatively may include the fact that a direct and/or indirect connection between the pieces of hardware is established by wired and/or wirelessly.

In an exemplary embodiment of the present disclosure, the vehicle may be referred to as being based on a concept including various means of transportation. In some cases, the vehicle may be interpreted as being based on a concept including not only various means of land transportation, such as cars, motorcycles, trucks, and buses, that drive on roads but also various means of transportation such as airplanes, drones, ships, etc.

For convenience in explanation and accurate definition in the appended claims, the terms “upper”, “lower”, “inner”, “outer”, “up”, “down”, “upwards”, “downwards”, “front”, “rear”, “back”, “inside”, “outside”, “inwardly”, “outwardly”, “interior”, “exterior”, “internal”, “external”, “forwards”, and “backwards” are used to describe features of the exemplary embodiments with reference to the positions of such features as displayed in the figures. It will be further understood that the term “connect” or its derivatives refer both to direct and indirect connection.

The term “and/or” may include a combination of a plurality of related listed items or any of a plurality of related listed items. For example, “A and/or B” includes all three cases such as “A”, “B”, and “A and B”.

In exemplary embodiments of the present disclosure, “at least one of A and B” may refer to “at least one of A or B” or “at least one of combinations of at least one of A and B”. Furthermore, “one or more of A and B” may refer to “one or more of A or B” or “one or more of combinations of one or more of A and B”.

In the present specification, unless stated otherwise, a singular expression includes a plural expression unless the context clearly indicates otherwise.

In the exemplary embodiment of the present disclosure, it should be understood that a term such as “include” or “have” is directed to designate that the features, numbers, steps, operations, elements, parts, or combinations thereof described in the specification are present, and does not preclude the possibility of addition or presence of one or more other features, numbers, steps, operations, elements, parts, or combinations thereof.

According to an exemplary embodiment of the present disclosure, components may be combined with each other to be implemented as one, or some components may be omitted.

The foregoing descriptions of specific exemplary embodiments of the present disclosure have been presented for purposes of illustration and description. They are not intended to be exhaustive or to limit the present disclosure to the precise forms disclosed, and obviously many modifications and variations are possible in light of the above teachings. The exemplary embodiments were chosen and described to explain certain principles of the present disclosure and their practical application, to enable others skilled in the art to make and utilize various exemplary embodiments of the present disclosure, as well as various alternatives and modifications thereof. It is intended that the scope of the present disclosure be defined by the Claims appended hereto and their equivalents.

What is claimed is:

1. An apparatus for determining occurrence of cyber sickness, the apparatus comprising:
a sensor unit configured to collect bio information of a user who experiences virtual-environment driving content, through a head-mounted display (HMD) and a vibration seat;
a first processing unit operatively connected to the sensor unit and configured to determine a first score by analyzing eye movements of the user using the bio information;
a second processing unit operatively connected to the sensor unit and configured to determine a second score by analyzing heart rate variability (HRV) of the user using the bio information;
a third processing unit operatively connected to the sensor unit and configured to determine a third score by analyzing a brainwave pattern of the user using the bio information; and
a fourth processing unit operatively connected to the sensor unit and configured to determine a cyber sickness index (CSI) of the user using the first score, the second score, and the third score.

2. The apparatus of claim 1, wherein the first processing unit is further configured to determine the first score by analyzing whether vergence of the user's eyes matches accommodation of the eyes.

3. The apparatus of claim 1, wherein the second processing unit is further configured to determine the second score by analyzing a heart rate, a heart rate pattern, and a standard deviation of NN intervals (SDNN) value of the user.

4. The apparatus of claim 1, wherein the third processing unit is further configured to determine the third score by analyzing frequency changes of alpha waves, theta waves, and delta waves of the user.

5. The apparatus of claim 1, wherein the fourth processing unit is further configured to determine the CSI by adding the first score, the second score, and the third score.

6. The apparatus of claim 5, wherein the fourth processing unit is further configured to determine score-specific contribution factors by performing regression analysis on the first score, the second score, and the third score.

7. The apparatus of claim 6, wherein the fourth processing unit is further configured to determine the CSI by applying the contribution factors as weights of the first score, the second score, and the third score.

8. The apparatus of claim 1, wherein the virtual-environment driving content includes a virtual image and a virtual sound generated based on a driving environment of a vehicle driver.

9. The apparatus of claim 8, wherein the virtual sound includes a high-order ambisonics signal.

10. The apparatus of claim 1, wherein the sensor unit includes:
a first sensor provided in the HMD to detect the eye movements of the user;
a second sensor provided in the vibration seat to measure a heart rate of the user; and
a third sensor provided in the HMD to detect brainwaves of the user.

11. A method of determining occurrence of cyber sickness, the method comprising:
collecting, by a sensor unit, bio information of a user who experiences virtual-environment driving content, through a head-mounted device (HMD) and a vibration seat;
determining, by a first processing unit operatively connected to the sensor unit, a first score by analyzing eye movements of the user using the bio information;
determining, by a second processing unit operatively connected to the sensor unit, a second score by analyzing heart rate variability (HRV) of the user using the bio information;
determining, by a third processing unit operatively connected to the sensor unit, a third score by analyzing a brainwave pattern of the user using the bio information; and
determining, by a fourth processing unit operatively connected to the sensor unit, a cyber sickness index (CSI) of the user using the first score, the second score, and the third score.

12. The method of claim 11, wherein the determining of the first score includes determining the first score by analyzing whether vergence of the user's eyes matches accommodation of the eyes.

13. The method of claim 11, wherein the determining of the second score includes determining the second score by analyzing a heart rate, a heart rate pattern, and a standard deviation of NN intervals (SDNN) value of the user.

14. The method of claim 11, wherein the determining of the third score includes determining the third score by analyzing frequency changes of alpha waves, theta waves, and delta waves of the user.

15. The method of claim 11, wherein the determining of the CSI includes determining the CSI by adding the first score, the second score, and the third score.

16. The method of claim 15, wherein the determining of the CSI includes determining score-specific contribution factors by performing regression analysis on the first score, the second score, and the third score.

17. The method of claim 16, wherein the determining of the CSI includes determining the CSI by applying the contribution factors as weights of the first score, the second score, and the third score.

18. The method of claim 11, wherein the virtual-environment driving content includes a virtual image and a virtual sound generated based on a driving environment of a vehicle driver.

19. The method of claim 18, wherein the virtual sound includes a high-order ambisonics signal.

20. The method of claim 11,
wherein the sensor unit includes a first sensor, a second sensor and a third sensor, and
wherein the collecting of the bio information includes:
detecting, by the first sensor provided in the HMD, the eye movements of the user;
measuring, by the second sensor provided in the vibration seat, a heart rate of the user; and
detecting, by the third sensor provided in the HMD, brainwaves of the user.
